(19) United States
(12) Patent Application Publication
(10) Pub. No.: US 2012/0232516 A1
(43) Pub. Date: Sep. 13, 2012

(54) DISPOSABLE PADDED UNDERWEAR

(75) Inventor: Albania Leon, Fort Lauderdale, FL (US)

(73) Assignee: Albania Leon, Fort Lauderdale, FL (US)

(21) Appl. No.: 13/417,216

(22) Filed: Mar. 10, 2012

Related U.S. Application Data
(60) Provisional application No. 61/451,410, filed on Mar. 10, 2011.

(51) Int. Cl. A61F 13/56

(52) U.S. Cl. 604/386

(57) ABSTRACT

The present invention is related to a disposable padded underwear comprising a thin disposable panty shape fabric defining a front portion, a back portion and an intermediate crotch portion therebetween, to said crotch portion a padded insert is secured in superposed relation forming a padded crotch surface, and the above mentioned front and back portions are attached by a removable attachment means.

### BACKGROUND OF THE INVENTION

1. Field of the Invention

The present invention relates generally to clothing and more particularly clothing suitable for use by women who desire to protect against staining or leakage from the flow of bodily fluids, The invention is, however, even more particularly directed to a disposable padded underwear that opens from the side for easy removal or wearing including an extra thick pad portion.

2. Description of the Prior Art

It is desirable to protect clothing and bedding against staining or leakage from a variety of sources that principally affects women. These sources include, for example, a bloody vaginal discharge that occurs with a menstrual flow or following childbirth, etc. Staining may also occur pursuant to medical treatment, where medicines are applied in the perineal area. Finally, incontinence is a relatively common problem among women of childbearing age and above. Staining of underwear is an irritating and sometimes costly problem experienced by many women, while the staining of outerwear or other items is an event that most women find profoundly embarrassing.

A variety of products are available to protect against various types of discharge or leakage. The most traditional include sanitary napkins and tampons. Sanitary napkins tend to be bulky and uncomfortable and prone to leakage. Further, the variety of conditions under which such items may be worn (generally referred to as “heavy flow” and “light flow”) means that no single product is likely to meet all needs. Tampons also have several disadvantages and have been shown to have an association with a menstrual toxic shock syndrome (TSS), a rare but sometimes fatal illness. Tampons may contain pesticides used on the cotton and chlorine which are used to bleach them. Some of the substances used to bleach tampons have been implicated in the formation of dioxin.

Disposable undergarments are very well known in the art. Most are directed toward hygienic panties having absorbent pads for menstrual use, incontinence, etc. The most disposable undergarments of the aforementioned type are bulky and require a large box or bag for carrying and are not lightweight or comfortable or otherwise suitable for everyday use.

There are also several disposable underwear known in the prior art. For example, a disposable hygienic panty comprising front and back panels of single layer non-woven cellulose material sewn together to form a waist portion and a body portion with leg openings. The waist band is formed by cloth strip sewn to the waist portion, and a cloth strip is sewn along the leg openings for reinforcing the edge of the material. Elastic bands are sewn into the cloth strips. A cotton gauze insert may be sewn into the crotch portion, or the panty may be completely lined with gauze.

Another known example is a disposable undergarment formed of a single sheet of non-woven fabric such as paper with thermoplastic material incorporated therein. The waist encircling portion is heat crimped in V-shaped corrugations to provide elasticity and stretch to the fabric and the side seams are heat fused together by melting the thermoplastic material.

Even though the above cited disposable underwear addresses some of the needs of the market, a disposable padded underwear that opens from the side for easy removal and wearing is still desired.

### SUMMARY OF THE INVENTION

This invention is directed to a disposable padded underwear that opens from the side for easy removal or wearing including an extra thick pad portion.

In one general aspect of the present invention, a disposable padded underwear comprises a thinner disposable panty portion, means for removable attachment and an extra thick pad portion for absorbency.

Furthermore, another aspect of this invention comprises a disposable padded underwear that it is easy to carry and use.

However, another aspect provides a disposable underwear which is inexpensive in construction and manufacture and which can be thrown away after use.

It is another object of this invention to provide a disposable underwear which is inexpensively constructed of attractive, soft, lightweight, comfortable, and durable material.

Another object of this invention is to provide a disposable underwear which is suitable for everyday use, and particularly useful for travelers and vacationers.

A further object of this invention is to provide a disposable underwear which occupies a very small space in its stored condition and will fit conveniently in a purse, pocket or suitcase.

In summary, the present invention is related to a disposable padded underwear comprising a thin disposable panty shape fabric defining a front portion, a back portion and an intermediate crotch portion therebetween, to said crotch portion a padded insert is secured in superposed relation forming a padded crotch surface, and the above mentioned front and back portions are attached by a removable attachment means.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a general perspective view of a disposable padded underwear in accordance with the present invention.

FIG. 2 is another perspective view of the present disposable padded underwear in which the means for removable attachment is shown in detail.

FIG. 3 is a front elevational view of one preferred embodiment of the disposable padded underwear in accordance with the present invention, finally:

FIG. 4 is a back elevational view another perspective view showing a child drinking from the carton and the straw inside the container.

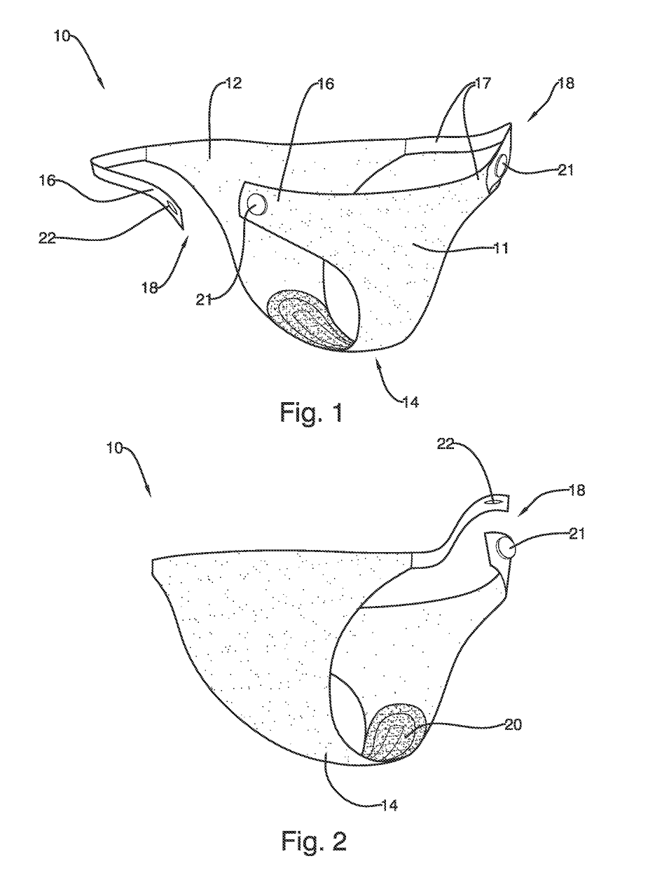

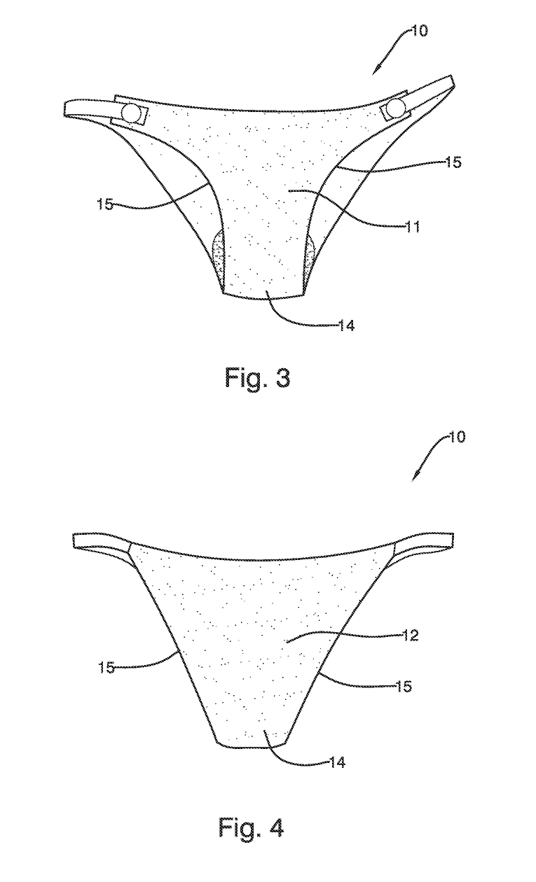

### DETAILED DESCRIPTION OF REPRESENTATIVE EMBODIMENTS

Shown throughout the Figures, the invention is directed to a disposable underwear 10 in the form of a lady's bikini panty. The underwear 10 is formed from a single piece of thin fabric material to form a front portion 11 and a back portion 12 with two generally semi-circular cut-outs 15 situated on each side of the longitudinal center line of the material to form the leg openings, and an intermediate crotch region 14.

Said generally rectangular crotch portion 14 having attached thereto a padded insert 20 which may be a commercially available sanitary napkin, a special padded fabric, or any material that creates a padded surface for absorbing bodily fluids. This insert 20 may be attached to the fabric using different solutions, including special glues, Velcro, seams, or the like.

The front and back portions 11-12 include an attachment means 18 at each side 16-17. In the illustrated embodiment said attachment means is a button 21 and a buttonhole 22 but this should not be interpreted as a limitation as any other attachment means may be used. For example, two pieces of Velcro sewn to the fabric may also be an adequate solution, or any other fast and easy attachment-detachment means that allows to the customer to take the pantry off easily. This is particularly useful when the customer must change her underwear during a trip, in a plane or any other situation in which she is away from home. It is also very practical when the user wears pants, as it is not even necessary to take her pants off to change the underwear.

Finally, for environmental reasons, it is possible to use biodegradable materials to manufacture the disposable padded underwear of the present invention.

While the preferred embodiments of the invention have been described above, it will be recognized and understood that various modifications can be made in the invention and the appended claims are intended to cover all such modifications which may fall within the spirit and scope of the invention

I claim:

1. Disposable padded underwear comprising a thin disposable panty shape fabric defining a front portion, a back portion and an intermediate crotch portion therebetween, to said crotch portion a padded insert is secured in superposed relation forming a padded crotch surface, and the above mentioned front and back portions are attached by a removable attachment means.
